(19) United States
(12) Patent Application Publication
(10) Pub. No.: US 2024/0398582 A1
(43) Pub. Date: Dec. 5, 2024

(54) GRAFT LOADING AND DEPLOYMENT SYSTEMS AND METHODS FOR ROTATOR CUFF REPAIR AND RECONSTRUCTION

(71) Applicant: ACTIVAS Orthopedics, Inc., Coronado, CA (US)

(72) Inventors: Colin Murphy, Carlsbad, CA (US); Brian Bowman, Carlsbad, CA (US); Benjamin Arnold, San Diego, CA (US); Thomas A. Afzal, Coronado, CA (US)

(21) Appl. No.: 18/679,041

(22) Filed: May 30, 2024

Related U.S. Application Data
(60) Provisional application No. 63/469,652, filed on May 30, 2023.

(51) Int. Cl. A61F 2/46; A61F 2/00

(52) U.S. Cl. CPC A61F 2/4612; A61F 2002/0072

(57) ABSTRACT

Systems, devices, and methods for deploying a graft to a treatment site of a patient. Exemplary systems include a device body and an insertion mechanism. The device body can have a handle and a trigger, and a controller. The insertion mechanism can be in operative association with the device body, and can have an outer sheath, a central shaft disposed at least partially within the outer sheath, an expansion mechanism in operative association with the central shaft, and a graft that can be deployed by the expansion mechanism.

### CROSS-REFERENCES TO RELATED APPLICATIONS

This application claims the benefit of U.S. Provisional Patent Application No. 63/469,652 filed May 30, 2023, the disclosure of which is incorporated herein by reference.

### BACKGROUND

Embodiments of the present invention relate generally to graft deployment systems and methods, and in particular instances, to graft deployment systems and methods for rotator cuff repair and reconstruction.

Current graft deployment modalities enable surgeons to provide beneficial treatments to patients in need thereof. Yet still further improvements in graft deployment technology is desired. Embodiments of the present invention provide solutions to at least some of these outstanding needs.

### BRIEF SUMMARY

Exemplary graft deployment systems or devices as disclosed herein can be provided as a single use device for the deployment of graft for rotator cuff reconstruction and rotator cuff repair. In some embodiments, the device can include a handle, a trigger, a collapsing feature, and an insertion mechanism. The insertion mechanism can include an outer sheath, a central shaft, and an expansion mechanism. The outer sheath can be made up of a distal sheath and a proximal sheath. The insertion mechanism can be loaded with graft by compressing the expansion mechanism with a loading rod, then loading the graft and distal sheath over the rod, which is then removed, leaving the loaded graft in place and the two parts of the outer sheath joined together. To deploy the graft the trigger is used to draw back the sheath and then expand the expansion mechanism. This presses the graft into position. The expansion mechanism can then be collapsed, and the insertion mechanism can be removed, leaving the graft in position for fixation.

Advantageously, embodiments of the present invention enable the performance of simplified surgical procedures by packaging full functionality into a single-use instrument, reducing cost associated with reprocessing. A simplified loading of the graft with the deployment tool can simplify manual preparation by the surgeon. In some cases, the expansion mechanism maintains rigidity after expansion, allowing for compression of the graft against target tissues, which is difficult with known solutions. In some cases, the expansion mechanism additionally allows for visualization and manipulation of the graft before fixation. Advantageously, in some embodiments the device can enable simplified procedures compared to some known solutions by minimizing need for suture.

Further, according to some embodiments, the semi-rigid nature of the expanded expansion mechanism allows for the ability to apply selective pressure to the graft during the fixation process, giving flexibility to the surgeon in terms of how they place fixation features. In some embodiments, a simple graft loading procedure removes the need for suture preparation, shortening and simplifying procedures. In some embodiments, the insertion mechanism allows for the positioning of the graft with visualization of its depth and location prior to full deployment. In some embodiments, advantages are provided by the configuration of a curved expansion mechanism. In some embodiments, advantages are provided by the use of a nitinol wire that is coiled in a sheet and curled.

In one aspect, embodiments of the present invention encompass systems and methods for deploying a graft for implantation. An exemplary device for deploying a graft for implantation includes a handle, a central shaft, an outer shaft, and an expansion mechanism. The central shaft can be coupled with the handle, and the central shaft can include a central post. The outer shaft can be configured to receive the graft at least partially therein, and the outer shaft can be configured to receive the central post at least partially therein such that the graft is at least partially wrapped around the central post. The expansion mechanism can be in operative association with the central shaft, and the expansion mechanism can include a wire that can be transitioned between a first state and a second state. In some cases, the handle is configured to actuate the outer shaft and the wire. In some cases, the device further includes a spring that operates to force the outer shaft in a proximal direction. In some cases, the device further includes a trigger, and the trigger can be operable to retract the outer shaft in a proximal direction. In some cases, the device includes a first mechanism operable to actuate the wire, and a second mechanism operable to actuate the outer shaft. In some cases, the device further includes a grip that operates to force the outer shaft in a proximal direction. In some cases, the handle is provided as a linear handle. In some cases, the handle is provided as a pistol grip. In some cases, the wire is made of or includes nitinol. In some cases, the outer shaft includes a proximal sheath and a distal sheath. In some cases, the distal sheath is configured to house the graft at least partially therein when the graft is in a curled configuration. In some cases, the distal sheath is configured to couple with the proximal sheath while housing the graft.

In another aspect, embodiments of the present invention encompass systems and methods for delivering a graft to a treatment site of a patient. An exemplary method of delivering a graft to a treatment site of a patient can include positioning the graft at least partially within an outer sheath of a graft deployment device, and placing the outer sheath at least partially over a central shaft of the graft deployment device, so that the graft is at least partially wrapped around a central post of the central shaft. The method can further include positioning the outer shaft at the treatment site of the patient while an expansion mechanism wire of the graft deployment device is in a first state, retracting the outer sheath and enabling the expansion mechanism wire to transition to a second state thereby expanding the graft, and pressing the graft against the treatment site of the patient. In some methods, the step of retracting the outer sheath and enabling the expansion mechanism wire to transition to the second state includes actuating a handle of the graft deployment device. In some methods, the step of retracting the outer sheath includes enabling a spring of the graft deployment device to force the outer shaft in a proximal direction. In some methods, the step of retracting the outer sheath includes actuating a trigger of the graft deployment device. In some methods, operating a first mechanism of the graft deployment device is effective to retract the outer sheath and operating a second mechanism of the graft deployment device is effective to enable the expansion mechanism wire to transition to the second state. In some methods, operating a grip of the graft deployment device is effective to force the outer shaft in a proximal direction. In some methods, the handle is provided as a linear handle. In some methods, the handle is provided as a pistol grip. In some methods, the wire is made of or includes nitinol. In some methods, the outer shaft of the graft deployment device includes a proximal sheath and a distal sheath. In some methods, the distal sheath is configured to house the graft at least partially therein when the graft is in a curled configuration. In some cases, methods can further include coupling the distal sheath with the proximal sheath while the distal sheath is housing the graft.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B illustrate aspects of a graft deployment device, according to embodiments of the present invention;

FIGS. 2A and 2B illustrate aspects of a graft deployment device, according to embodiments of the present invention;

FIGS. 3A and 3B illustrate aspects of a graft deployment device, according to embodiments of the present invention;

FIGS. 4A and 4B illustrate aspects of a handle of a graft deployment device, according to embodiments of the present invention;

FIGS. 5A and 5B illustrate aspects of a trigger of a graft deployment device, according to embodiments of the present invention;

FIGS. 6A to 6C illustrates aspects of an outer sheath of a graft deployment device, according to embodiments of the present invention;

FIGS. 7A to 7C illustrate aspects of a graft deployment device, according to embodiments of the present invention;

FIGS. 8A to 8C illustrate aspects of a graft deployment device, according to embodiments of the present invention;

FIGS. 9A to 9C illustrate aspects of a graft deployment system, according to embodiments of the present invention;

FIGS. 10A and 10B illustrate aspects of a graft deployment system, according to embodiments of the present invention;

FIGS. 11A to 11C illustrate aspects of a graft deployment system, according to embodiments of the present invention;

FIGS. 12A and 12B illustrate aspects of a graft deployment system, according to embodiments of the present invention;

FIGS. 13A and 13B illustrate aspects of a graft deployment system, according to embodiments of the present invention;

FIGS. 14A and 14B illustrates aspects of a graft deployment method, according to embodiments of the present invention;

FIGS. 15A and 15B illustrates aspects of a graft deployment method, according to embodiments of the present invention;

FIGS. 16A and 16B illustrates aspects of a graft deployment method, according to embodiments of the present invention; and

FIGS. 17A and 17B illustrate aspects of a graft deployment method, according to embodiments of the present invention.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

Graft deployment systems disclosed herein can be used to deploy grafts to any of a variety of patient treatment sites. In some cases, a graft deployment system can be used to deploy a graft to a rotator cuff of a patient. In some cases, a graft deployment system can be used to deploy a graft for an Achilles tendon repair, a patella tendon repair, a triceps repair, a quadriceps repair, or the like. In some embodiments, graft deployment systems or methods disclosed herein may incorporate one or more aspects of the systems or methods which are described in U.S. patent application Ser. No. 18/382,899 filed Oct. 23, 2023, the content of which is incorporated herein by reference.

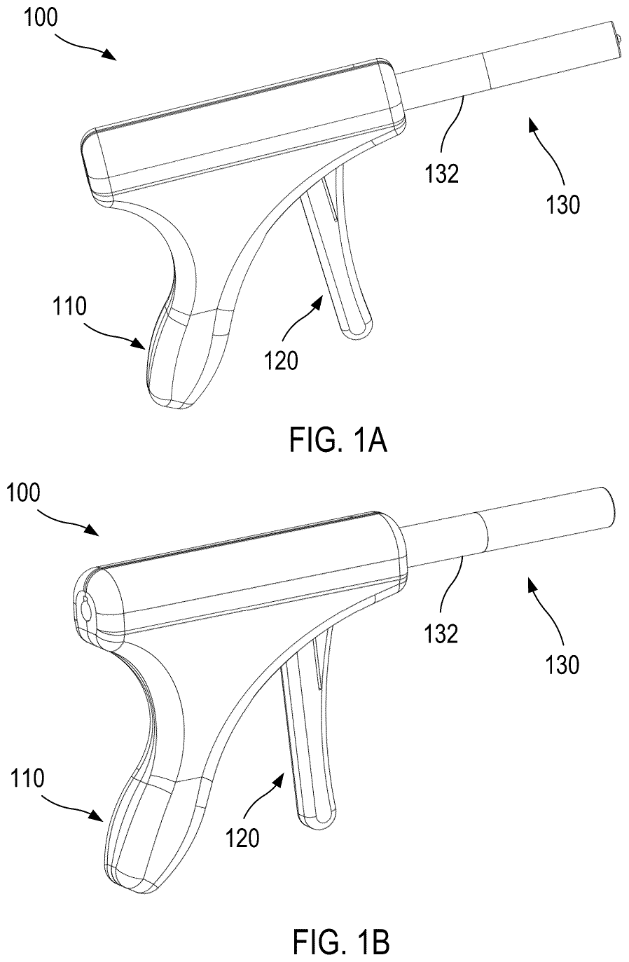

Turning now to the drawings, FIGS. 1A and 1B depict aspects of a graft deployment device 100, according to embodiments of the present invention. As shown here, graft deployment device 100 includes a handle 110, a trigger 120, and an insertion mechanism 130. In some cases, the insertion mechanism 130 can be provided as a collapsing assembly or feature. In some cases, the insertion mechanism can include an outer sheath 132. The outer sheath 132 can be provided as an assembly of multiple parts, or as a single member. In some embodiments, the handle 110 can be provided with a pistol grip orientation. In some embodiments, the handle can be provided with a linear orientation. In some cases, the handle 110 can include the trigger 120. In some cases, the handle can be configured to actuate the outer sheath or shaft 132. In some cases, the handle can be configured to actuate aspects of the insertion mechanism (e.g. an expansion mechanism or one or more components thereof). In some embodiments, the handle 110 can be provided as a grip that is operable to actuate (e.g. retract) the outer sheath 132. In some embodiments, the trigger 120 is operable to actuate (e.g. retract) the outer sheath 132. In some cases, the handle and the trigger can collectively be referred to as a body.

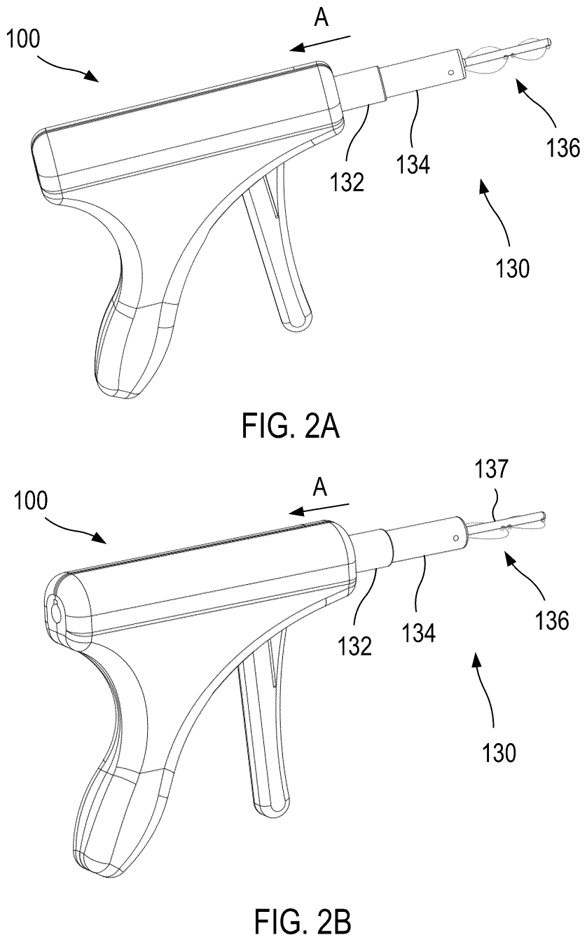

FIGS. 2A and 2B depict further aspects of a graft deployment device 100. As shown here, an insertion mechanism 130 can include an outer shaft 132, a central shaft 134, and an expansion mechanism or feature 136. In some cases, expansion mechanism 136 can be referred to as an expansion assembly. Extending from the central shaft 134 is a central post 137. In the views provided by FIGS. 2A and 2B, the outer shaft 132 has been retracted proximally in the direction indicated by arrow A to reveal the central shaft 134 and central post 137. In some embodiments, the expansion mechanism 136 can include a looped wire. In some embodiments, the expansion mechanism 136 can include a looped nitinol wire. In some cases, an expansion mechanism can include openings for visualization or fixation. In some embodiments, an expansion mechanism can be provided having a shape designed to conform to patient anatomy.

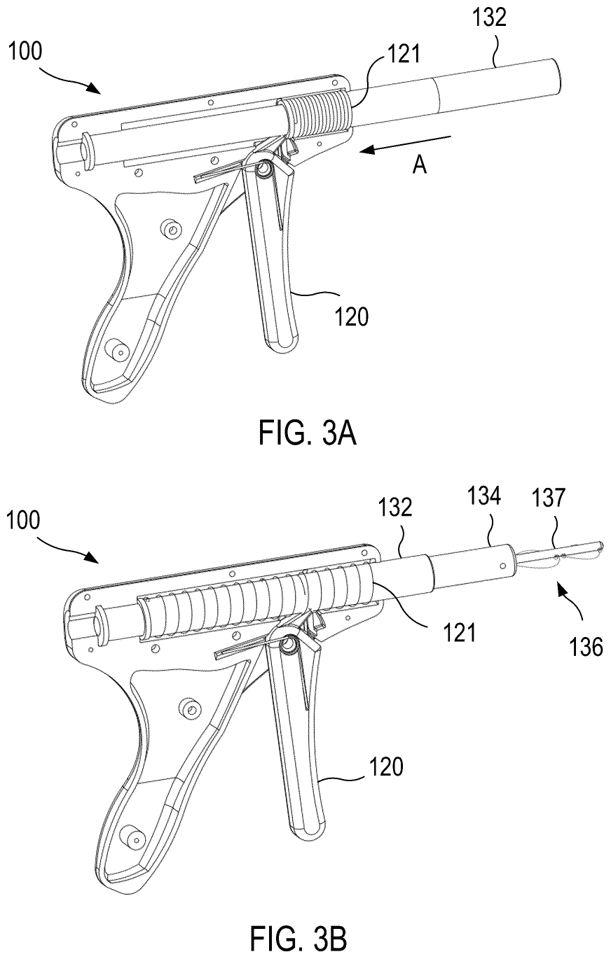

FIGS. 3A and 3B provide cross-section views of aspects of a graft deployment device 100. As shown here, the trigger 120 can be actuated to enable the outer shaft 132 to retract proximally in the direction indicated by arrow A, and thereby expose the central shaft 134 and central post 137. In some embodiments, the device 100 includes a coil spring 121 that expands when the trigger 120 is actuated, thereby forcing the outer shaft 132 in a proximal direction. In some embodiments, a device may include another mechanism which operates to retract the outer shaft. In some cases, the device 100 includes a first mechanism operable to actuate the expansion mechanism 136 (e.g. a collapsing mechanism), and a second mechanism (e.g. a trigger) operable to actuate the outer shaft 132.

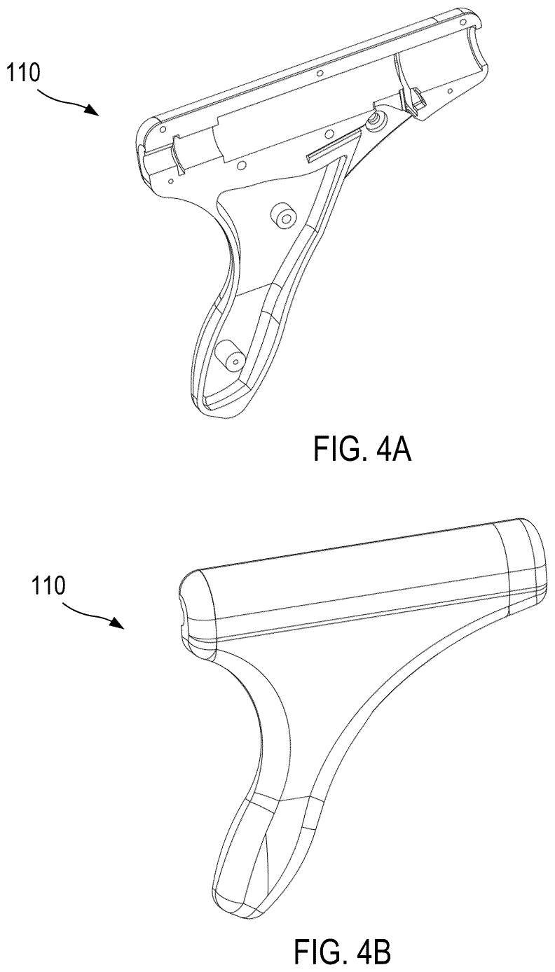

FIGS. 4A and 4B depict aspects of a handle 110 of a graft deployment device.

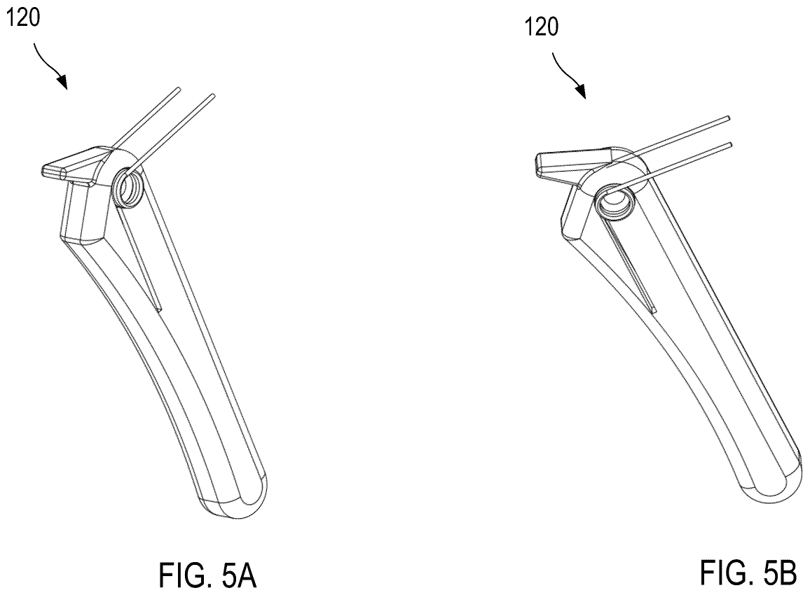

FIGS. 5A and 5B depict aspects of a trigger 120 of a graft deployment device.

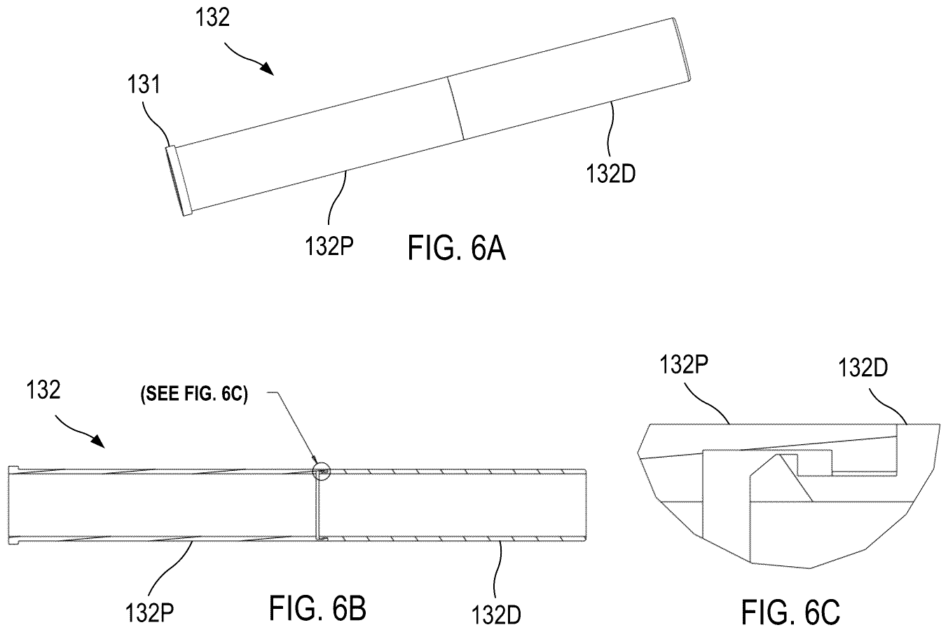

FIGS. 6A to 6C illustrate aspects of an outer shaft or sheath 132. As shown here, an outer shaft or sheath 132 can include a proximal outer shaft or sheath 132P and a distal outer shaft or sheath 132D. As shown in the enlarged view provided by FIG. 6C, the proximal outer shaft or sheath 132P and the distal outer shaft or sheath 132D can be coupled via a snap fit connection. In some cases, the snap fit connection is a releasable snap fit connection. In some cases, the proximal outer shaft or sheath 132P and the distal outer shaft or sheath 132D can be coupled together with some other connection means. The proximal outer sheath 132P includes a proximal flange 131 that can engage a spring (e.g. so that the spring can force the sheath 132 in a proximal direction when the spring is actuated), as discussed elsewhere herein.

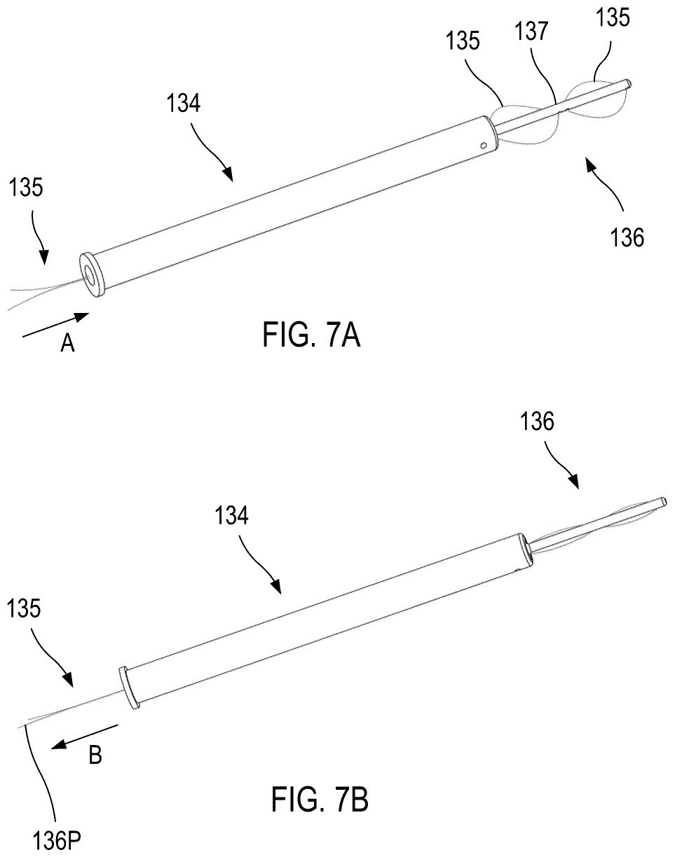

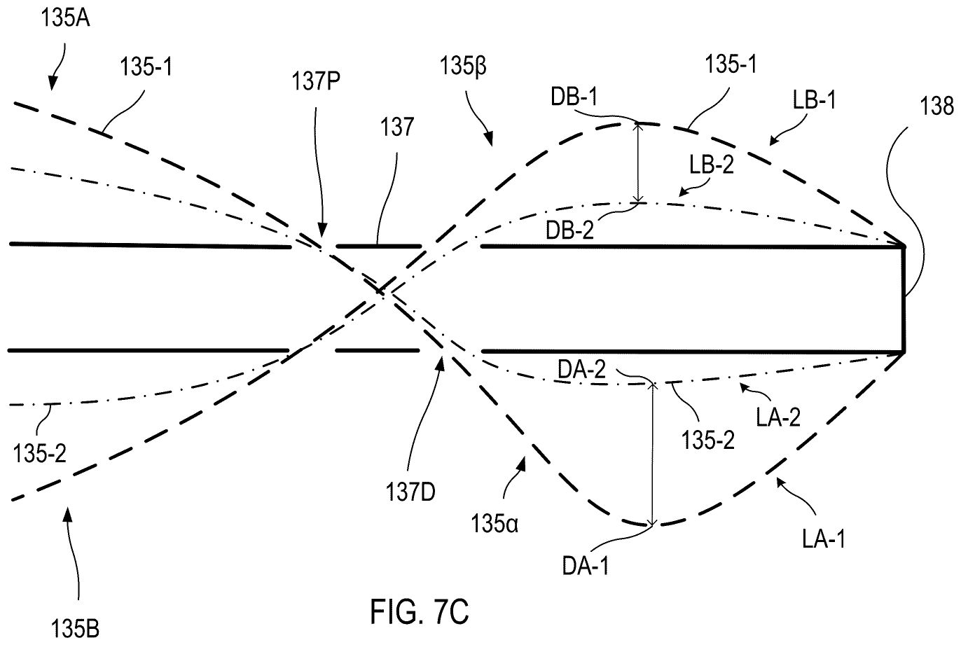

FIGS. 7A to 7C depict additional aspects of a graft deployment device. As shown here, the expansion assembly or feature 136 can transition between a first or expanded state as shown in FIG. 7A and a second or collapsed state as shown in FIG. 7B. In some embodiments, the expansion assembly 136 can include or be in operative association with a collapsing mechanism 135 such as a wire, which can include or be provided as a nitinol wire. In some cases, the collapsing mechanism 135 is at least partially disposed within the central shaft 134. In some cases, the expansion assembly 136 can be converted from a collapsed state to an expanded state by pushing on the collapsing member 135 in a distal direction as indicated by arrow A. In some cases, the expansion assembly 136 can be converted from an expanded state to a collapsed state by drawing on the collapsing member 135 in a proximal direction as indicated by arrow B. In some cases, the collapsing member 135 is made of a shape memory material such as nitinol which in an uncompressed or relaxed state orients to the first state shape shown in FIG. 7A and that can be compressed to the second state shape shown in FIG. 7B. In some cases, a collapsing mechanism 135 can be referred to as a collapsing member. In some cases, a collapsing mechanism or member can include one or more wires, threads, filaments, or the like. In some cases, a collapsing mechanism can refer to a portion 136A of an expansion assembly that can be grasped or otherwise manipulated to convert the expansion assembly from an expanded state to a collapsed state.

In the cross-section side view provided in FIG. 7C, it can be seen that the central post 137 includes a distal channel or aperture 137D and a proximal channel or aperture 137P through which sections of the collapsing mechanism 135 may pass. As shown here, the collapsing member 135 can adopt a first or expanded state 135-1 as well as a second or collapsed state 135-2. In the embodiment depicted here, the collapsing member 135 includes a first wire portion 135A that is coupled with a distal end 138 of the central post 137, and a second wire portion 135B that is coupled with a distal end 138 of the central post 137.

The portion 135α of the first collapsible member component 135A that extends between the distal aperture 137d and the distal end 138 can have a length LA-1 when the collapsible member is in the first state and a length LA-2 when the collapsible member is in the second state, where length LA-1 is greater than LA-2. Relatedly, the portion 135% of the first collapsible member component 135A that extends between the distal aperture 137d and the distal end 138 can have an arc height DA-1 when the collapsible member is in the first state and an arc height DA-2 when the collapsible member is in the second state, where the arc height DA-1 is greater than the arc height DA-2. The arc height can refer to the distance between the central post 137 and the maxima of the portion 135α of the first collapsible member component 135A that extends between the distal aperture 137d and the distal end 138. So, for example, the maxima moves between distance DA-1 and DA-2 when the first collapsible member component 135A moves between the first state and the second state.

Likewise, the portion 135β of the second collapsible member component 135B that extends between the distal aperture 137d and the distal end 138 can have a length LB-1 when the collapsible member is in the first state and a length LB-2 when the collapsible member is in the second state, where length LB-1 is greater than LB-2. Relatedly, the portion 135β of the second collapsible member component 135B that extends between the distal aperture 137d and the distal end 138 can have an arc height DB-1 when the collapsible member is in the first state and an arc height DB-2 when the collapsible member is in the second state, where the arc height DB-1 is greater than the arc height DB-2. The arc height can refer to the distance between the central post 137 and the maxima of the portion 135 of the second collapsible member component 135B that extends between the distal aperture 137D and the distal end 138. So, for example, the maxima moves between distance DB-1 and DB-2 when the second collapsible member component 135B moves between the first state and the second state.

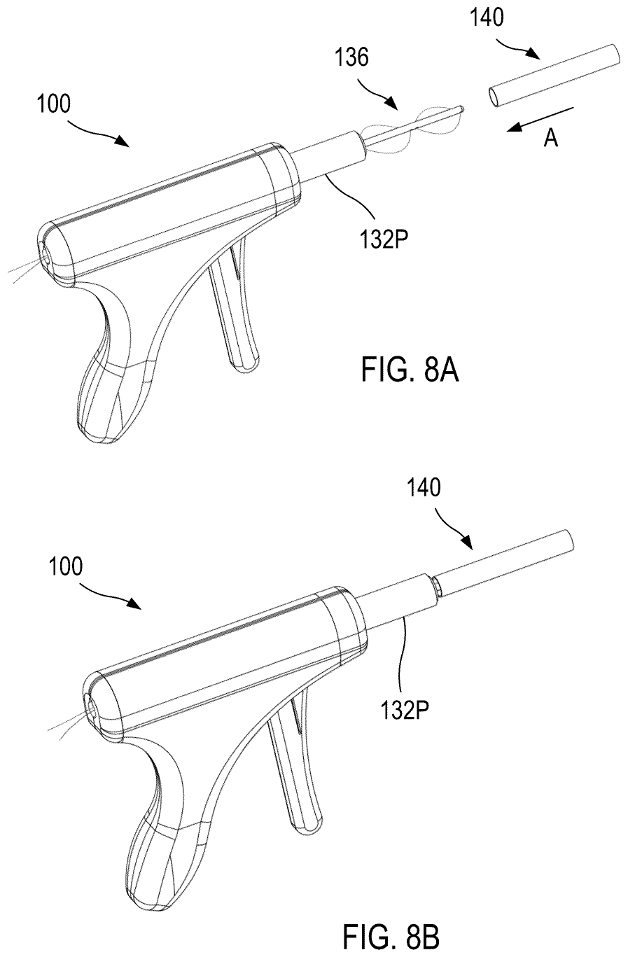

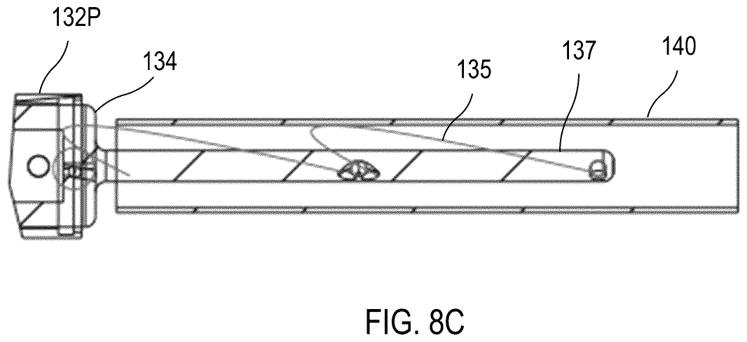

FIGS. 8A to 8C show aspects of a graft deployment procedure, according to embodiments of the present invention. As shown in FIGS. 8A and 8B, a loading rod 140 can be inserted or placed over the expansion assembly 136, for example by advancing the loading rod 140 in a proximal direction as indicated by arrow A. As shown in the enlarged view provided by FIG. 8C, the loading rod 140 operates to compress or constrain the collapsing member or wire 135. Hence, in some embodiments, the collapsing member or wire 135 folds back in a proximal direction as the loading rod or shaft 140 is slid over it. In this image, the central shaft 134 is at least partially nested or positioned within the proximal outer shaft or sheath 132P.

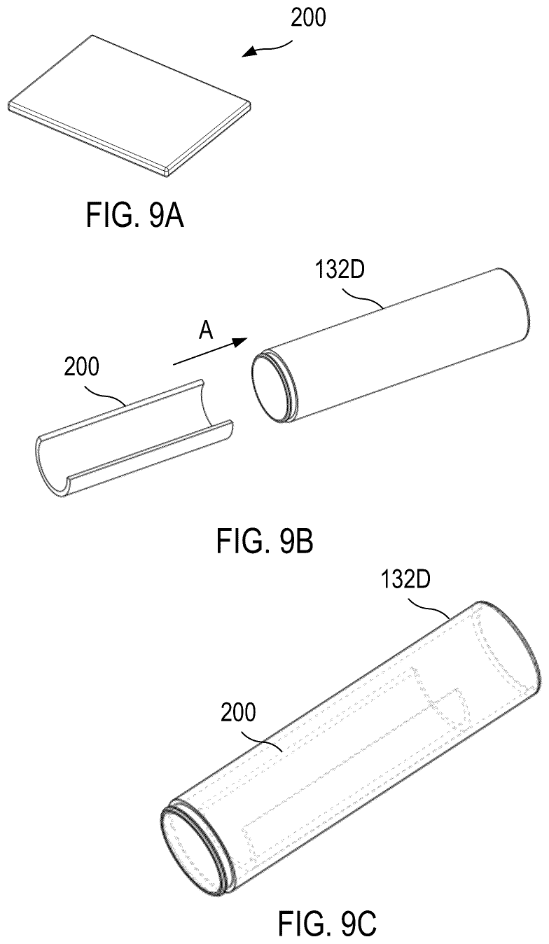

FIGS. 9A to 9C show aspects of a graft deployment procedure, according to embodiments of the present invention. FIG. 9A depicts a graft 200. As shown in FIG. 9B, the graft 200 can be deformed (e.g. rolled from a flattened shape into a half-cylindrical or semi-cylindrical shape) and placed or advanced into the distal outer shaft or sheath 132D as indicated by arrow A. FIG. 9C depicts the graft 200 disposed within the distal outer shaft or sheath 132D.

Hence, to load the system, a graft 200 can be first prepared per the surgeon's preference. The graft 200 can then be loaded onto the inside of the distal sheath 132D. As discussed elsewhere herein, a loading rod can be placed over an expansion mechanism in order to put the expansion mechanism into a compressed state. The distal sheath 132D and graft 200 can then be inserted or placed over the loading rod until the distal sheath 132D snaps onto a proximal sheath, joining the two sheath components into one outer sheath. The loading rod can then be removed and the device can be loaded. In some embodiments, the graft 200 can be loaded with suture. In some embodiments, grafts can be provided in different sizes. In some cases, devices can be provided in various sizes according to the required sizes of the graft. In some cases, a graft can be trimmed as needed.

In some repair embodiments, a graft can have a size of about 20-25 mm by about 25-30 mm and 1-3 mm thick, and an outer sheath can have an outer diameter of about 10 mm. In some reconstruction embodiments, a graft can have a size of about 40 by 75 mm and 3-5 mm thick, and an outer sheath can have an outer diameter of about 12 mm or more.

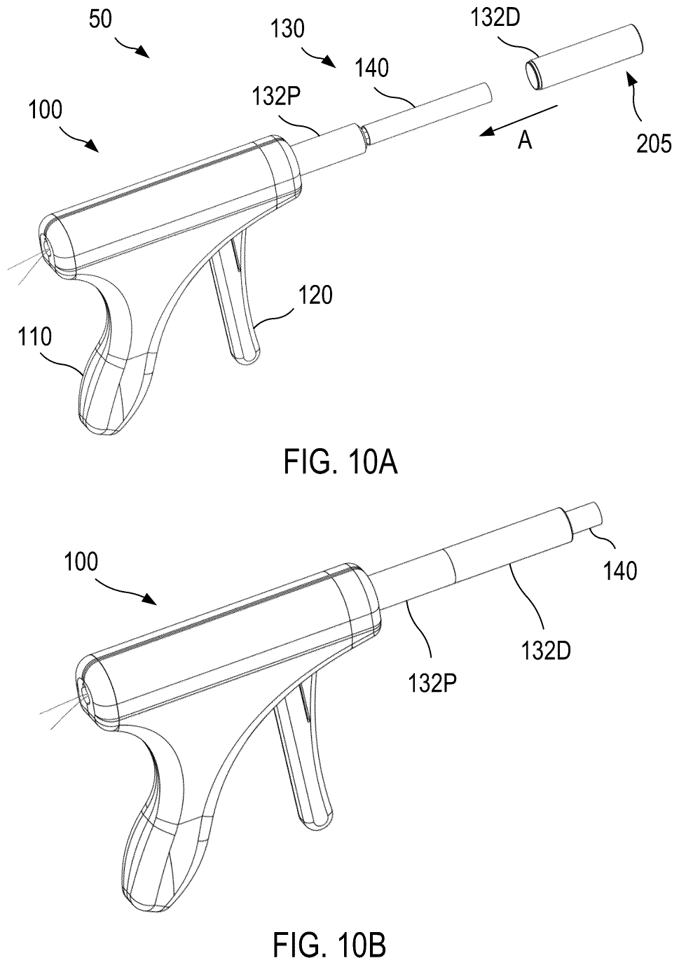

FIGS. 10A and 10B show aspects of a graft deployment procedure, according to embodiments of the present invention. As depicted here, a combination 205 of the graft (not visible) disposed within the distal outer shaft or sheath 132D can be loaded over the loading rod 140, for example by moving the combination in a proximal direction as indicated by arrow A, and the distal outer shaft or sheath 132D can be coupled with the proximal outer shaft or sheath 132P, for example by a snap fit connection as previously described in reference to FIG. 6C. A distal portion of the loading rod 140 can extend from the distal outer shaft or sheath 132D, and this distal portion of the loading rod can be grasped when the user or operator wishes to remove the loading rod 140 from the device 100.

In some embodiments, a graft deployment system 50 includes a graft deployment device 100, a loading rod 140, and a graft (not visible). The graft deployment device 100 can include a handle 110, a trigger 120, a collapsing feature or mechanism 136, and an insertion mechanism 130. The insertion mechanism 130 can include an outer sheath having a proximal section 132P and a distal section 132D, a central shaft (not visible), and an expansion mechanism (not visible). In some cases, the outer sheath is made up of a proximal sheath that has a snap feature capable of joining with a corresponding snap feature on the distal sheath. The central shaft and the outer sheath can be fixated in the handle 110, with the central shaft rigidly fixed and the outer sheath spring loaded but otherwise free to move axially when not blocked by the trigger 120. The trigger 120 can be attached to the handle 110 such that it can be pulled comfortably with the same hand holding the handle, and such that it initially blocks the movement of the outer sheath. The expansion mechanism can be located on the distal end of the central shaft. In some cases, a collapsing mechanism can include or be provided as a switch or a knob. In some cases, a collapsing mechanism could be used in place of a loading rod.

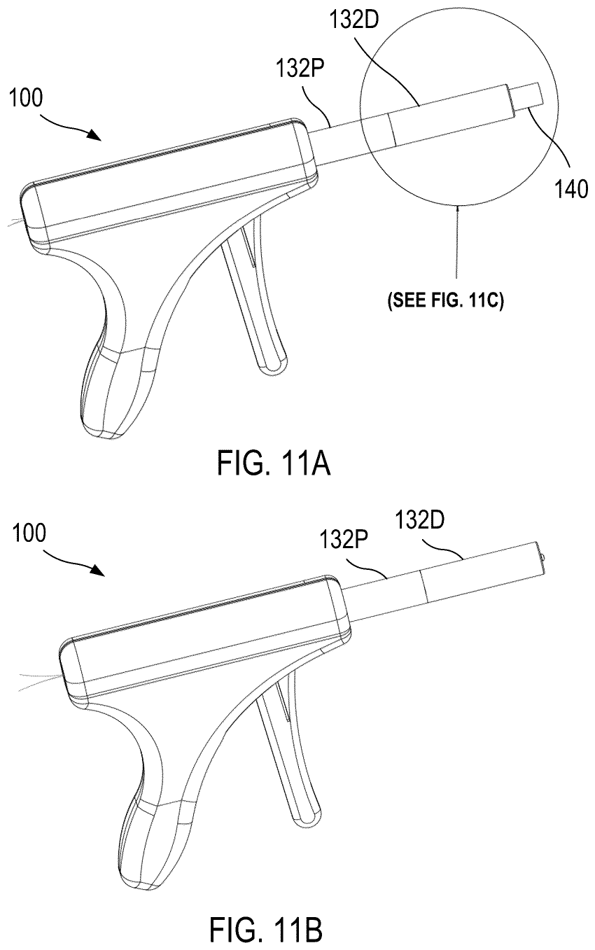

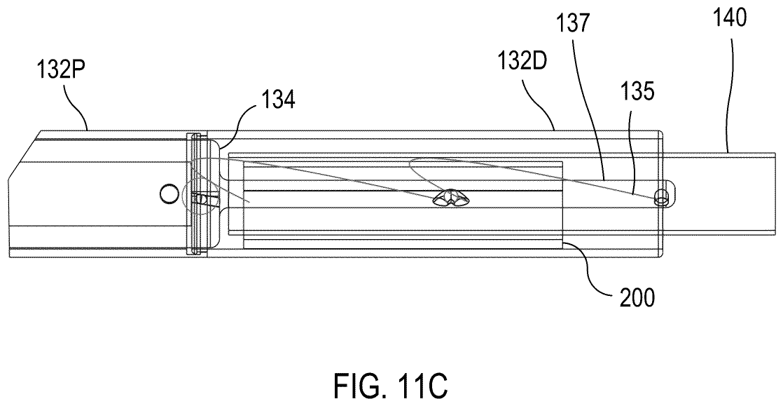

FIGS. 11A to 11C show aspects of a graft deployment procedure, according to embodiments of the present invention. FIG. 11C depicts the location of the graft 200 as it resides within the distal outer shaft or sheath 132D. Hence, the insertion mechanism can be loaded with a graft by compressing the expansion mechanism with a loading rod, then loading the graft and distal sheath over the rod, which is then removed, leaving the loaded graft in place and the two parts of the outer sheath joined together.

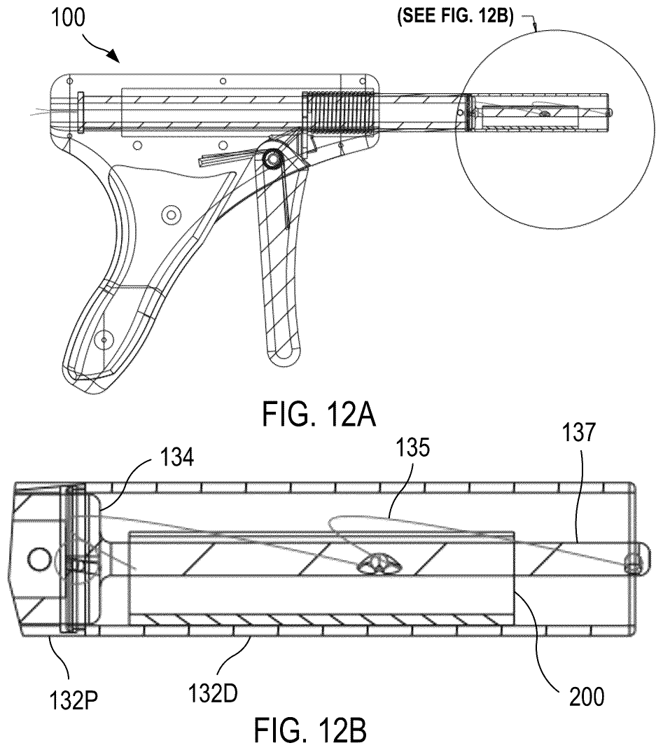

FIGS. 12A and 12B show aspects of a graft deployment procedure, according to embodiments of the present invention. As depicted in FIG. 12B, the loading rod has been removed, and the collapsing member or wire 135 is no longer constrained by it. Hence, the insertion mechanism can be loaded with a graft by compressing the expansion mechanism with a loading rod, then loading the graft and distal sheath over the rod, which is then removed, leaving the loaded graft in place and the two parts of the outer sheath joined together.

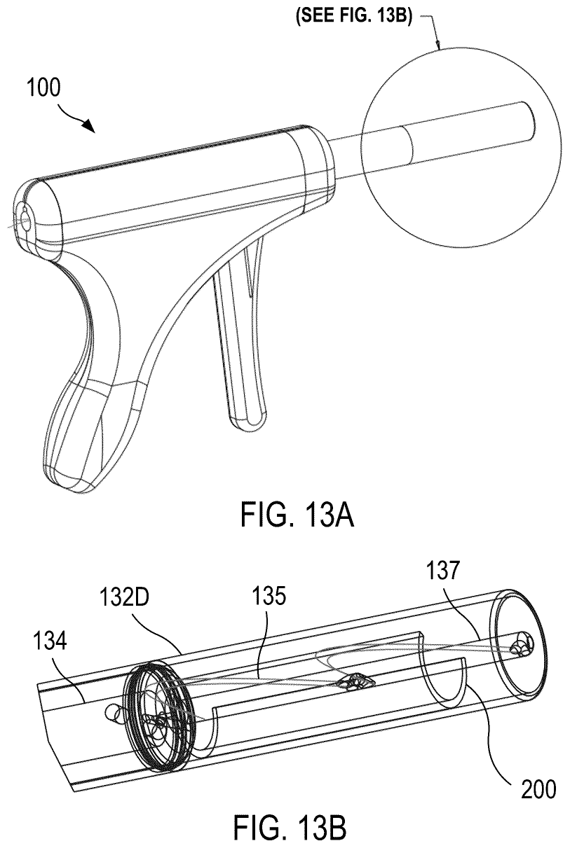

FIGS. 13A and 13B show aspects of a graft deployment procedure, according to embodiments of the present invention. As depicted in FIG. 13B, the loading rod has been removed, and the collapsing member or wire 135 is no longer constrained by it. Hence, the insertion mechanism can be loaded with a graft by compressing the expansion mechanism with a loading rod, then loading the graft and distal sheath over the rod, which is then removed, leaving the loaded graft in place and the two parts of the outer sheath joined together.

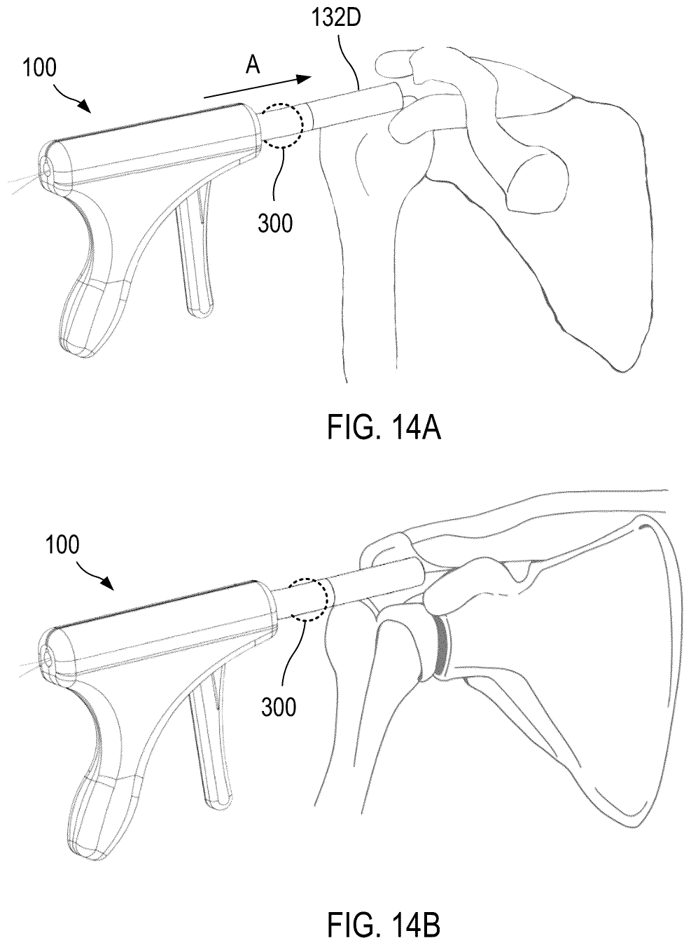

FIGS. 14A and 14B show aspects of a graft deployment procedure, according to embodiments of the present invention. As illustrated here, the distal outer shaft or sheath 132D can be advanced through a port hole 300 as indicated by arrow A, and positioned at a desired location to which the graft can be delivered or deployed.

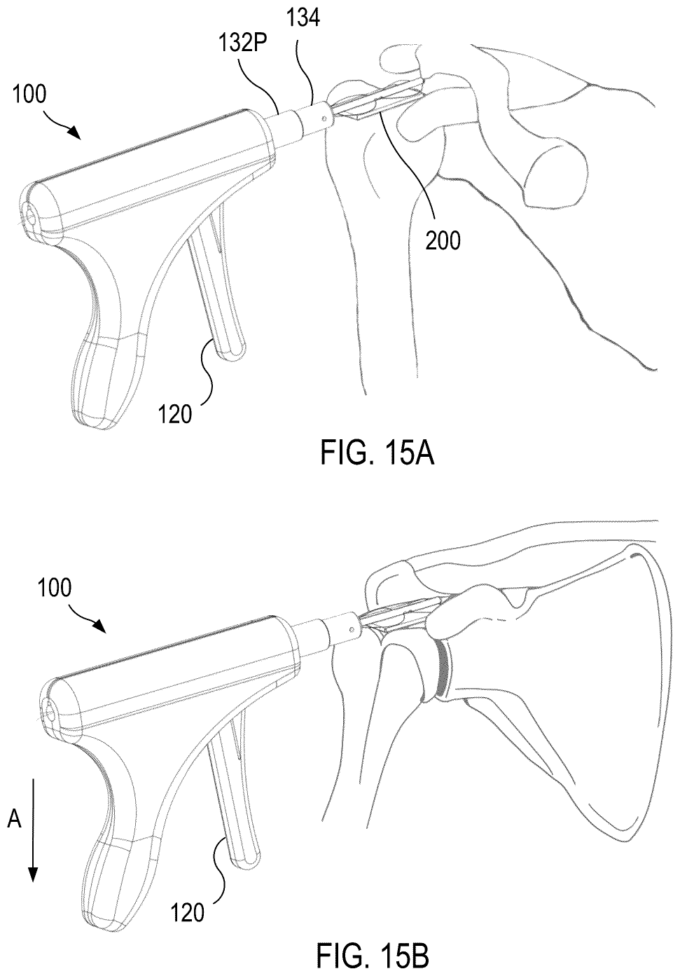

FIGS. 15A and 15B show aspects of a graft deployment procedure, according to embodiments of the present invention. As illustrated here, the distal outer shaft or sheath has been removed, the collapsible member component has transitioned from the collapsed state to the expanded state, and the graft 200 has been positioned at the patient anatomy. In operation the user or operator can pull the trigger 120 while maintaining a downward pressure on the device 100 as indicated by arrow A. Hence, to deploy the graft the trigger can be used to draw back the sheath and then expand the expansion mechanism. This can press the graft into position. The expansion mechanism can then be collapsed, and the insertion mechanism can be removed, leaving the graft in position for fixation.

Hence, the deployment device 100 can be used by situating the distal end of the outer sheath at the location where the graft is desired, then pulling the trigger 120. The outer sheath is drawn back, revealing the graft 200 and expansion mechanism. The expansion mechanism flattens the graft onto the surface (e.g. surface of tissue) for grafting. The graft 200 can then be fixated as desired. Once ready, the collapsing mechanism can be used to collapse the expansion mechanism. For example, with reference to FIG. 7B, an expansion assembly 136 can be converted from an expanded state to a collapsed state by drawing on the collapsing mechanism 135 in a proximal direction as indicated by arrow B. The deployment device can then be removed.

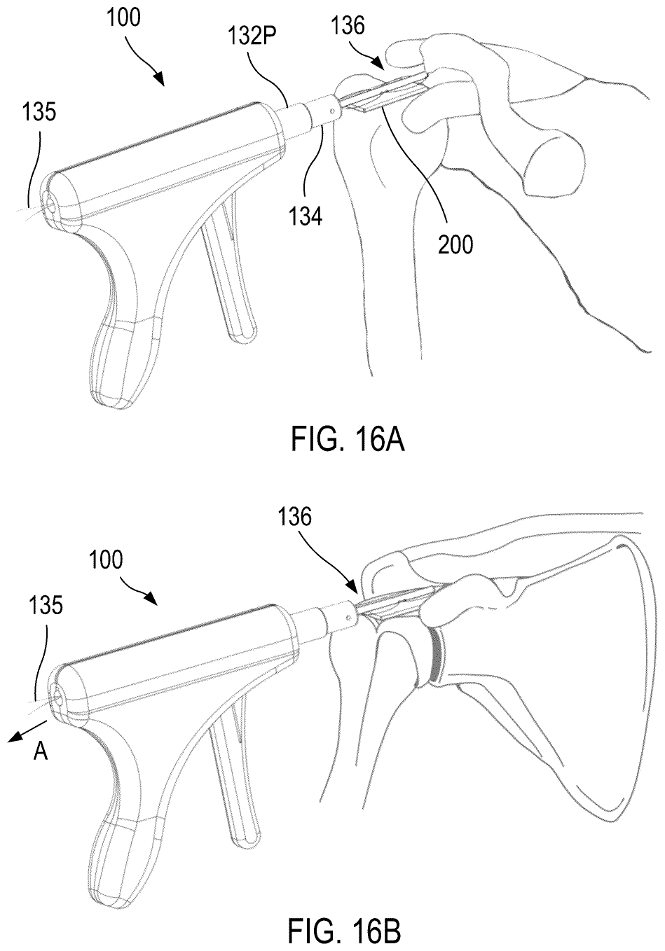

FIGS. 16A and 16B show aspects of a graft deployment procedure, according to embodiments of the present invention. As illustrated here, the user or operator can retract the collapsing member or wire 135 in a proximal direction as indicated by arrow A, which can operate to collapse the expansion mechanism 136. Hence, to deploy the graft the trigger can be used to draw back the sheath and then expand the expansion mechanism. This can press the graft into position. The expansion mechanism can then be collapsed, and the insertion mechanism can be removed, leaving the graft in position for fixation.

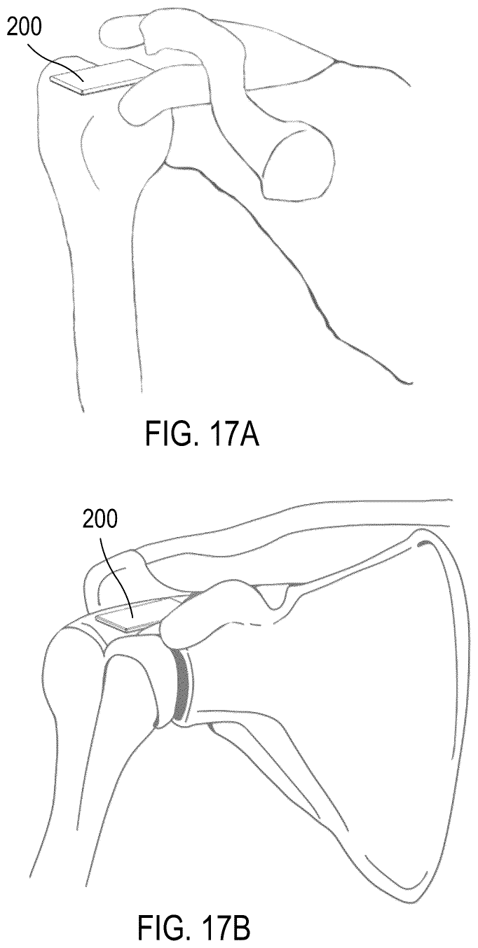

FIGS. 17A and 17B show aspects of a graft deployment procedure, according to embodiments of the present invention. As shown here, the graft 200 has been deployed in the desired anatomical location of the patient.

Hence, use of a graft deployment system can involve advancing an insertion mechanism through a portal, toward the desired or proper position. The insertion mechanism can be inserted to a desired depth of the graft and positioned so that the center of the graft is at the center of the desired graft location. Once the desired location is reached, the trigger can be pulled, retracting the outer sheath. In some cases, the trigger can be pulled a second time to expand the expansion mechanism. The expansion mechanism, now expanded, can be used to tamp down the graft, and to hold the graft in place while fixation is applied to the graft (e.g. to a distal end of the graft). Once sufficient fixation is applied, the lever can be used to compress the expansion mechanism, and the graft deployment system can be removed from the patient.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for deploying a graft for implantation, the device comprising:
a handle;
a central shaft coupled with the handle, the central shaft comprising a central post;
an outer shaft, wherein the outer shaft is configured to receive the graft at least partially therein, and wherein the outer shaft is configured to receive the central post at least partially therein such that the graft is at least partially wrapped around the central post;
an expansion mechanism in operative association with the central shaft, the expansion mechanism comprising a wire that can be transitioned between a first state and a second state.

2. The device according to claim 1, wherein the handle is configured to actuate the outer shaft and the wire.

3. The device according to claim 1, wherein the device further comprises a spring that operates to force the outer shaft in a proximal direction.

4. The device according to claim 1, wherein the device further comprises a trigger, and the trigger is operable to retract the outer shaft in a proximal direction.

5. The device according to claim 1, wherein the device includes a first mechanism operable to actuate the wire, and a second mechanism operable to actuate the outer shaft.

6. The device according to claim 1, wherein the device further comprises a grip that operates to force the outer shaft in a proximal direction.

7. The device according to claim 1, wherein the handle is provided as a linear handle.

8. The device according to claim 1, wherein the handle is provided as a pistol grip.

9. The device according to claim 1, wherein the wire comprises nitinol.

10. The device according to claim 1, wherein the outer shaft comprises a proximal sheath and a distal sheath, wherein the distal sheath is configured to house the graft at least partially therein when the graft is in a curled configuration, and wherein the distal sheath is configured to couple with the proximal sheath while housing the graft.

11. A method of delivering a graft to a treatment site of a patient, the method comprising:
positioning the graft at least partially within an outer sheath of a graft deployment device;
placing the outer sheath at least partially over a central shaft of the graft deployment device, so that the graft is at least partially wrapped around a central post of the central shaft;
positioning the outer shaft at the treatment site of the patient while an expansion mechanism wire of the graft deployment device is in a first state;
retracting the outer sheath and enabling the expansion mechanism wire to transition to a second state thereby expanding the graft; and
pressing the graft against the treatment site of the patient.

12. The method according to claim 11, wherein the step of retracting the outer sheath and enabling the expansion mechanism wire to transition to the second state comprises actuating a handle of the graft deployment device.

13. The method according to claim 11, wherein the step of retracting the outer sheath comprises enabling a spring of the graft deployment device to force the outer shaft in a proximal direction.

14. The method according to claim 1, wherein the step of retracting the outer sheath comprises actuating a trigger of the graft deployment device.

15. The method according to claim 1, wherein operating a first mechanism of the graft deployment device is effective to retract the outer sheath and operating a second mechanism of the graft deployment device is effective to enable the expansion mechanism wire to transition to the second state.

16. The method according to claim 1, wherein operating a grip of the graft deployment device is effective to force the outer shaft in a proximal direction.

17. The method according to claim 12, wherein the handle is provided as a linear handle.

18. The method according to claim 12, wherein the handle is provided as a pistol grip.

19. The method according to claim 1, wherein the wire comprises nitinol.

20. The method according to claim 3, wherein the outer shaft of the graft deployment device comprises a proximal sheath and a distal sheath, wherein the distal sheath is configured to house the graft at least partially therein when the graft is in a curled configuration, and wherein the method further comprises coupling the distal sheath with the proximal sheath while the distal sheath is housing the graft.
